(19) United States
(12) Patent Application Publication
(10) Pub. No.: US 2005/0177130 A1
(43) Pub. Date: Aug. 11, 2005

(54) Balloon catheter with spiral folds

(75) Inventors: Eitan Konstantino, Orinda, CA (US); Tanhum Feld, Moshav (IL)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: ANGIOSCORE, INC., Alameda, CA (US)

(21) Appl. No.: 10/776,457

(22) Filed: Feb. 10, 2004

(51) Int. Cl. A61M 31/00

(52) U.S. Cl. 604/509; 606/194; 264/138; 425/391

(57) ABSTRACT

The balloon catheter comprises a radially expansible, polymeric balloon having one or more permanent helical fold lines so that the balloon can be spirally folded upon delivery of the catheter to or removal from a vessel. A scoring structure may be carried over the balloon in between the folds to score a stenosed region in a blood vessel. Fabrication devices and methods for spirally folding the balloon are also disclosed.

### CROSS REFERENCES TO RELATED APPLICATIONS

This application is related to 60/442,161 (Attorney Docket No. 021770-000100US), filed on Jan. 21, 2003, and Ser. No. 10/631,499 (Attorney Docket No. 021770-000110US) filed on Jul. 30, 2003, the full disclosures of which are incorporated herein by reference.

### BACKGROUND OF THE INVENTION

1. Field of the Invention

The present invention relates to the field of medical devices, more specifically to medical devices intended to treat stenoses in the vascular system.

Balloon dilatation (angioplasty) is a common medical procedure mainly directed at revascularization of stenotic vessels by inserting a catheter having a dilatation balloon through the vascular system. The balloon is inflated inside a stenosed region in a blood vessel in order to apply radial pressure to the inner wall of the vessel and widen the stenosed region to enable better blood flow.

In many cases, the balloon dilatation procedure is immediately followed by a stenting procedure where a stent is placed to maintain vessel patency following the angioplasty. Failure of the angioplasty balloon to properly widen the stenotic vessel, however, may result in improper positioning of the stent in the blood vessel. If a drug-eluting stent is used, its effectiveness may be impaired by such improper positioning and the resulting restenosis rate may be higher. This is a result of several factors, including the presence of gaps between the stent and the vessel wall, calcified areas that were not treated properly by the balloon, and others.

Conventional balloon angioplasty suffers from a number of other shortcomings as well. In some cases the balloon dilatation procedure causes damage to the blood vessel due to aggressive balloon inflation that may stretch the diseased vessel beyond its elastic limits. Such over inflation may damage the vessel wall and lead to restenosis of the section that was stretched by the balloon. In other cases, slippage of the balloon during the dilatation procedure may occur. This may result in injury to the vessel wall surrounding the treated lesion. One procedure in which slippage is likely to happen is during treatment of in-stent restenosis, which at present is difficult to treat by angioplasty balloons. Fibrotic lesions are also hard to treat with conventional balloons, and elastic recoil is usually observed after treatment of these lesions. Many long lesions have fibrotic sections that are difficult to treat using angioplasty balloons.

An additional problem with conventional angioplasty balloon design has been delivery and extraction of the balloon within the vessel. It is desirable for the deflated balloon to have a small a profile as possible and increase the balloon flexibility in order to improve the catheter performance in many aspects; i.e. improve ability to cross tight lesions, and improve pushability and overall deliverability to minimize trauma to the vessel. Traditional folding procedures and/or designs generally fold the balloon straight and parallel to the catheter axis. When the balloon having this configuration is collapsed for removal of the balloon, the balloon collapses in a random manner, typically leaving the balloon in a “pancake” shape having a cross section that is flat in one direction and wide in a second direction. After the inflation medium is removed from a balloon, the deflated configuration will often have a width greater than the original folded configuration which was introduced to the vasculature. Such an increase in profile can make removal of the balloon difficult, and cause trauma to the vessel. Where angioplasty is performed on multiple sites in the vessel, the problem becomes even more prevalent.

Another problem associated with balloon angioplasty treatment has been the “watermelon seed effect.” Angioplasty is carried out at very high pressures, typically up to twenty atmospheres or higher, and the radially outward pressure of the balloon can cause axial displacement of the balloon in a manner similar to squeezing a watermelon seed with the fingers. Such axial displacement, of course, reduces the effectiveness of balloon dilatation.

For these reasons, it would be desirable to provide improved balloon folding designs and methods for their manufacture. In particular it would be desirable to provide a balloon which folds to form a small profile for delivery, and readily permits deflation to a folded state having the same or similar configuration and profile as it had prior to inflation to facilitate removal from the vasculature. At least some of these objectives will be met with the inventions described hereinafter.

2. Description of the Background Art

The following U.S. patents and printed publication relate to cutting balloons and balloon structures: U.S. Pat. Nos. 6,450,988; 6,425,882; 6,394,995; 6,355,013; 6,245,040; 6,210,392; 6,190,356; 6,129,706; 6,123,718; 5,891,090; 5,797,935; 5,779,698; 5,735,816; 5,624,433; 5,616,149; 5,545,132; 5,470,314; 5,320,634; 5,221,261; 5,196,024; and Published U.S. Pat. App. Nos. 2003/0153870 and 2003/0032973. Other U.S. patents of interest include U.S. Pat. Nos. 6,454,775; 5,100,423, 4,998,539; 4,969,458; and 4,921,984.

### BRIEF SUMMARY OF THE INVENTION

The present invention provides improved apparatus and methods for the dilatation of stenosed regions in the vasculature. The stenosed regions will often include areas of fibrotic, calcified, or otherwise hardened plaque or other stenotic material of the type which can be difficult to dilatate using conventional angioplasty balloons. The methods and apparatus will often find their greatest use in treatment of the arterial vasculature, particularly the coronary arterial vasculature, but may also find use in treatment of the venous and/or peripheral vasculature, treatment of small vessels and/or vessel bifurcations that will not be stented, treatment of ostial lesions, and treatment of ISR.

In one aspect of the invention, a balloon catheter comprises a catheter body having a proximal end and a distal end, and a radially expansible balloon near the distal end of the catheter body, the balloon comprising a proximal end, a distal end, and at least one helical fold line formed on the balloon prior to folding, which extends helically along at least a portion of the surface of the balloon. The radially expansible balloon may have any number of helical fold lines, but preferably has between two and five helical folds. The fold lines may be parallel and equally spaced apart from each other. The fold lines may comprise grooves, score lines, creases, recesses, channels, etc. on the outside surface of the balloon, and the balloon can be folded over along the preformed fold lines to form lobes or flaps spirally extending across the balloon.

In some embodiments, the catheter further comprises a scoring structure adjacent to the fold lines. The scoring structure generally has at least one scoring element spirally circumscribing the balloon. The scoring element may continuously circumscribe the balloon or comprise a plurality of segments. The scoring element may have a variety of configurations, often being a thin structure in the form of a wire or slotted tube having a circular, square, or other cross-sectional geometry. Preferably, the scoring elements will comprise a scoring edge, either in the form of a honed blade, a square shoulder, or the like. In some embodiments, the scoring structure will be located within a recess extending helically across at least a portion of the surface of the balloon.

In all cases, the scoring structure is preferably composed of an elastic material, more preferably a super elastic material, such as nitinol, stainless steel or a combination thereof. Generally, the scoring element is secured to an outer surface of the balloon between the lobes or flaps so that the scoring element is shielded by the lobes or flaps to prevent contact between the scoring element and the vessel walls at both delivery and removal of the catheter. When the balloon is inflated, the scoring structure elastically expands over the expanded balloon. Upon deflation, the scoring structure will elastically close to its original non-expanded configuration, thus helping to close and contain the balloon.

In another embodiment of the invention, a method of folding a balloon on a balloon catheter for insertion in a body lumen comprises: providing a balloon having at least one fold line formed in a wall of the balloon and extending helically along the outer surface of the balloon; and folding the balloon along the at least one helical fold line. Generally, the catheter is inserted in its helically-folded state into a body lumen, advanced to a treatment site within the lumen, and the balloon is inflated to engage a wall of the lumen to treat the lumen. After treatment, the balloon is deflated so that the folds collapse into the helically compressed state, wherein the catheter is either advanced to another treatment site or removed from the lumen.

In many embodiments, the at least one fold line is created by scoring the balloon. In one method of the invention, scoring the balloon comprises advancing the balloon relative to a fixture having at least one scoring element, and scoring the balloon to create a score that extends helically along an outer surface of the balloon. Typically, the balloon is advanced is advanced relative to a stationary fixture. Alternatively, the fixture may be advanced relative to a stationary balloon.

Generally, the fixture has between two and five scoring elements, and preferably between three and four scoring elements. The scoring elements may comprise any means for permanently creating a fold line in the outer surface of the balloon, for example scoring blades, lasers, ultrasonic emitters, radiofrequency (RF) emitters, or resistive heaters.

In many embodiments the fixture further comprises an opening, wherein the scoring blades are positioned to converge on the opening, and the balloon is advanced through the opening to score the balloon. Typically, the scoring blades individually rotate about a mounting axis of each scoring blade as the balloon is advanced through the opening. In many aspects, the balloon is rotated as it is advanced through the opening. Alternatively, the scoring blades may be canted so as to force the balloon to rotate as it is advanced through the opening of the fixture. In general, the scoring blades are heated and temperature controlled by a heating means.

In another method of the invention, folding the balloon along the helical fold line is performed by inserting the balloon into a press. Preferably, the method further comprises heating the press. In some embodiments, the balloon is inflated to a predetermined pressure prior to advancing the balloon relative to a fixture, and the balloon is deflated at a predetermined rate as it is being scored. Typically, the balloon is inflated to a range between 15 psi and 400 psi and is deflated at a rate in the range between 1 psi/sec and 150 psi/sec.

In one aspect of the invention, a method is disclosed for creating the at least one fold line by permanently creasing the balloon. The permanent crease may be created by advancing a portion of the balloon into a press comprising a plurality of helical folding plates each having a mating helical surface positioned adjacent to each other. A portion of the balloon is positioned between the mating helical surfaces, and the plates are pressed together to form at least one fold line extending helically along an outside surface of the balloon. The press generally comprises two to five folding plates to create one or more of helical fold lines.

In some embodiments, the press comprises an expandable support, wherein the folding plates are held adjacent to each other by the expandable support. Prior to advancing the balloon into the press, the expandable support may be sufficiently expanded to allow the balloon to be positioned between the mating surfaces of the helical folding plates. The plates may then be pressed together by compressing the expandable support.

In another aspect of the invention, the at least one helical fold line is created by heating a portion of the balloon in a helical pattern. One method of heating the balloon comprises: providing an expansible cage having at least one helical segment, wherein the number of helical segments corresponds to the number of helical fold lines to be fabricated; inflating the balloon inside the cage so that the balloon contacts the at least one helical segment; heating the cage; and radially compressing the cage while deflating the balloon to create at least one helical fold line along the at least one helical segment. The cage generally has two to five helical segments to create the helical fold lines. Typically, the helical segments comprise wire. Additionally, the method may further comprise inserting the compressed cage and balloon into a tube having an inner diameter such that the balloon is folded over onto the helical fold line. Alternatively, the method may comprise rolling the balloon so that the balloon folds helically along the fold line.

In another aspect of the invention, a device is disclosed for fabricating a radially expansible balloon for a balloon catheter having at least one helical fold. The device generally comprises a base connected to a mounting fixture adapted to receive the balloon, a means for advancing the balloon relative to the mounting fixture, and at least one scoring element mounted to the mounting fixture. In typical operation, the number of scoring elements corresponds to the number of helical folds to be fabricated, and the at least one scoring element converges on the balloon as it is advanced so that the scoring element creates at least one score extending helically along the outer surface of the balloon, thus allowing the balloon to be helically folded along the score.

In some cases the fixture has between two and five scoring elements, and preferably between three and four scoring elements. The scoring elements can have a variety of particular configurations, and may comprise any means for permanently creating a fold line in the outer surface of the balloon, for example scoring blades, lasers, ultrasonic emitters, radiofrequency (RF) emitters, or resistive heaters.

Where scoring blades are used, the fixture further comprises an opening. The scoring blades are positioned to converge on the opening so that the balloon may be advanced through the opening to score the balloon. Often, the scoring blades comprise discs rotatably mounted on the fixture, wherein the discs roll along the outside surface of the balloon as it is advanced past the opening. This configuration allows the balloon to be scored without scraping the surface of the balloon.

In a preferred embodiment, the scoring blades are canted so as to force the balloon to rotate as it is advanced through the opening of the fixture. This rotation causes the scoring elements to create helical fold lines as the balloon is advanced through the opening. Alternatively, the fixture is rotatably mounted to the base, and the base further comprises a means for rotating the fixture at a predetermined rate. In this configuration, the balloon is pulled axially through the fixture at a predetermined speed, thereby creating helical fold lines in the balloon as it is advanced through the rotating fixture. Preferably, the scoring blades are heated by a heating means so that the fold lines are permanently engraved into the balloon. The heating means may comprise any heat source such as a resistive heating element, RF energy, ultrasonic energy, or the like. The heating source may also provide means for controlling and maintaining the temperature of the scoring blades.

The device generally has a means for advancing the balloon catheter relative to the fixture. The advancement means may push or pull the balloon catheter along its axis into the fixture. Generally, the advancement means comprises a linear actuator to advance the balloon at a predetermined rate. However, the advancement means may comprise any means for axially displacing the along its axis, such as manual insertion by hand. The advancement may also allow for the balloon to rotate about the balloon axis as it is being inserted into the fixture. Alternatively, the advancement means may comprise a separate rotation means for rotating the balloon at a predetermined rate as it is being axially displaced.

In yet another embodiment, a device is disclosed for fabricating a radially expansible balloon for a balloon catheter having at least one helical fold. The device has a plurality of helical plates each having mating helical surface. The mating helical surfaces are placed adjacent to each other to form a at least one helical gap. The device also comprises a support for the holding the helical plates together. Preferably, the support is adjustable to permit the size of the at least one helical gap to be modified. The device further has means for applying pressure to press the helical plates together, wherein a portion of the balloon may be inserted between the helical plates and pressed to form one or more fold lines extending helically along an outer surface of the balloon. Usually, adjusting the support provides means for applying pressure to the press. The device also comprises means for heating the plates to permanently form the helical fold lines.

In yet another embodiment, a device for fabricating a radially expansible balloon for a balloon catheter having at least one helical fold comprises an expansible cage having at least one helical segment, a heat source coupled to the cage wherein the heat source heats the cage to create the at least one helical fold line. The number of helical segments corresponds to the number of helical fold lines to be fabricated, typically from two to five helical segments. In Most embodiments, the caged is configured to fit around the circumference of the balloon so that the helical segments contact the balloon when the balloon is in an inflated configuration, and wherein the cage collapses with the balloon when the balloon is deflated. The cage may also function as a scoring structure that is delivered with the balloon catheter for treatment of a body lumen.

### BRIEF DESCRIPTION OF THE DRAWINGS

FIG. 1 illustrates a balloon catheter having helical flaps folded in a compressed configuration in accordance with the present invention.

FIG. 2 illustrates a balloon catheter having helical lobes in a compressed configuration in accordance with the present invention.

FIG. 3 illustrates a balloon catheter having a scoring cage in between helical lobes in accordance with the present invention.

FIG. 4 is view of the device of FIG. 1 in an non-folded configuration.

FIG. 5 is another view of the device of FIG. 1 in a folded configuration.

FIG. 6 is an enlarged front view of the device of FIG. 1 in a folded configuration.

FIG. 7 illustrates a balloon catheter having a scoring cage in between helical flaps in accordance with the present invention.

FIG. 8 is an illustration of the device of FIG. 7 in a folded configuration.

FIG. 9 is an enlarged front view of the device of FIG. 7 in a folded configuration.

FIG. 10a is a schematic illustration of a balloon catheter in a compressed configuration delivered to a treatment region in a vessel in accordance with the present invention.

FIG. 10b is a schematic illustration of a balloon catheter in an expanded configuration delivered to a treatment region in a vessel in accordance with the present invention.

FIG. 11 a schematic illustration of a fixture for fabricating helical fold lines on a balloon catheter in accordance with embodiments of the invention.

FIG. 12 is an enlarged view of the aperture of the fixture of FIG. 11.

FIG. 13 a schematic illustration of another fixture for fabricating helical fold lines on a balloon catheter in accordance with embodiments of the invention.

FIG. 14 is a cross-sectional view of the fixture of FIG. 13.

FIG. 15 is an enlarged view of the aperture the fixture of FIG. 13.

FIG. 16 is a schematic illustration of another fixture for fabricating helical fold lines on a balloon catheter in an expanded configuration in accordance with embodiments of the invention.

FIG. 17 is an illustration of the fixture of FIG. 13 in a compressed configuration.

### DETAILED DESCRIPTION OF THE INVENTION

In the following description, various aspects of the present invention will be described. For purposes of explanation, specific configurations and details are set forth in order to provide a thorough understanding of the present invention. However, it will also be apparent to one skilled in the art that the present invention may be practiced without the specific details presented herein. Furthermore, well-known features may be omitted or simplified in order not to obscure the present invention.

Embodiments of the present invention relate to device for revascularization of stenotic vessels and specifically to a balloon catheter having helical folds. The balloon catheter comprises a conventional dilatation balloon such as a radially expansible, polymeric balloon having permanent helical fold lines so that the balloon can be spirally folded upon insertion or removal of the catheter from a vessel.

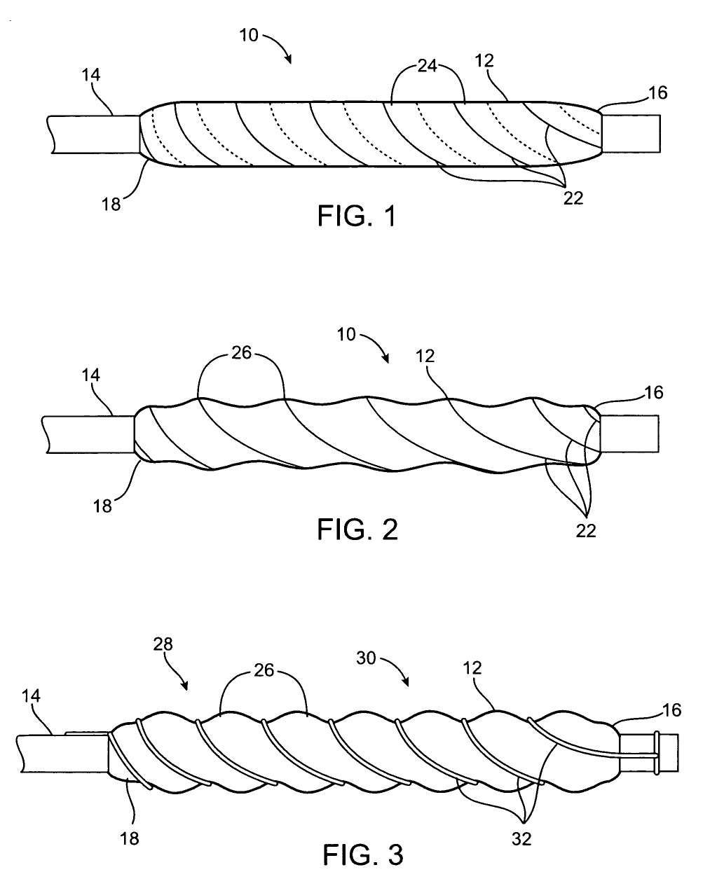

Reference is now made to FIGS. 1 and 4-6, which are illustrations of a balloon catheter 10 in accordance with embodiments of the invention. The balloon catheter 10 includes a radially expansible dilatation balloon 12, which may be any conventional angioplasty balloon such as commonly used by interventional cardiologists or radiologists. The balloon 12 is disposed at or near the distal end of catheter body 14. Balloon 12 has at least one permanent helical or spiral fold line 22 emanating from the distal tip 16 of the balloon to the proximal end extending helically across the balloon until it terminates at the proximal end 18 of the balloon.

The radially expansible balloon 12 may have any number of helical fold lines 22, but generally has between two and five fold lines, and preferably between three and four fold lines. The fold lines 22 are generally parallel and equally spaced apart from each other as shown in FIG. 1, but may comprise any helical pattern. The fold lines 22 may comprise grooves, score lines, creases, recesses, channels, or other demarcations that are preformed on the outside surface of the balloon, and the balloon can be folded over along the preformed fold lines to form lobes or flaps spirally extending across the balloon.

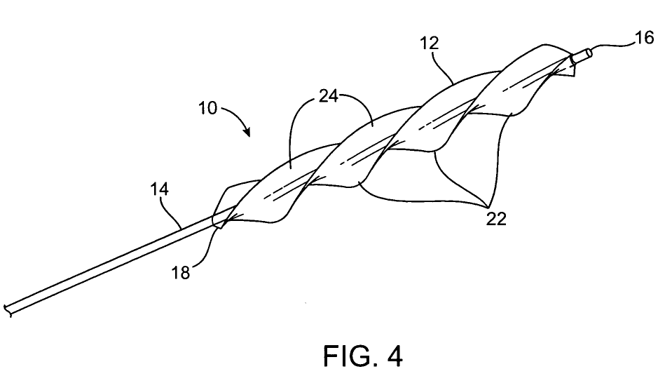

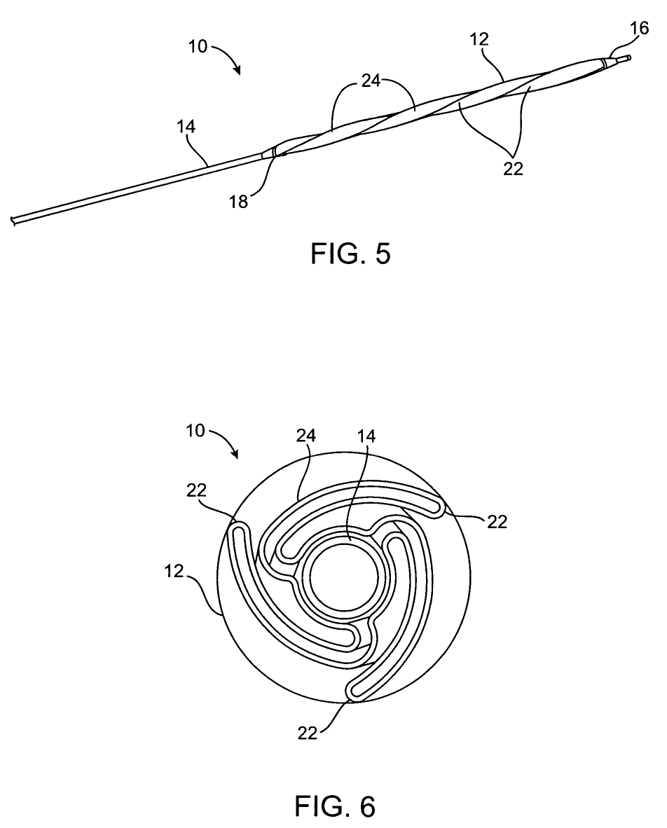

Referring to FIG. 4, the balloon may be depressurized to form helical flaps 24 extending coincident with the helical fold lines 22 from the distal end 16 of the balloon to the proximal end 18 of the balloon. The embodiment as illustrated in FIGS. 4-6 has three helical flaps extending across the length of the balloon. As seen in FIGS. 5 and 6, the three helical flaps 24 may be folded down to press against the catheter body 14 so that the profile of the balloon 12 is minimized for insertion the balloon catheter into and removal from the vessel.

FIG. 2 illustrates an alternative embodiment of the present invention wherein a balloon catheter 20 comprises a balloon 12 that is folded upon the plurality of helical fold lines 22 to form helical lobes 26 running coincident with the fold lines and extending from the distal end 16 of the balloon the proximal end 18 of the balloon. When balloon 12 of catheter 20 is depressurized, the helical lobes compress against the catheter body 14 into a low-profile configuration.

Referring to FIG. 3, another embodiment of the invention comprises a balloon catheter 30 having a radially expansible dilatation balloon 12, and a helical or spiral scoring structure 28 mounted over or attached to balloon 12. Preferably, the scoring structure 28 is secured to an outer surface of the balloon 12 and runs adjacent to the helical fold lines 22 and between the helical lobes 26 so that the scoring structure is shielded by the lobes to prevent contact between the scoring structure and the vessel walls at both delivery and removal of the catheter.

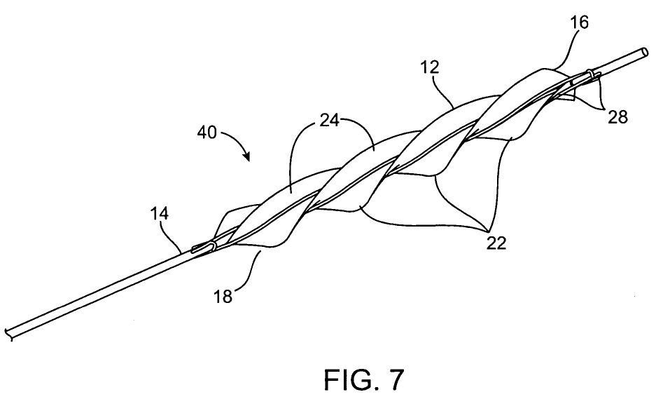

Alternatively, scoring structure 28 may be disposed in between the helical flaps 24 of the balloon catheter 40 shown in FIG. 7. When the helical flaps 24 of the balloon 12 are folded down onto the catheter body 14, the scoring structure 28 is shielded by the flaps to prevent contact between the scoring structure and the vessel walls during delivery of the balloon catheter.

In some embodiments, the scoring structure 28 may be attached at its proximal and distal ends to the proximal end 18 and distal end 16 of balloon 12. Alternatively, the scoring structure 28 may be attached at its proximal and distal ends to catheter body on either side of the proximal end 18 and distal end 16 of balloon 12.

The scoring structure 28 generally has at least one scoring element 32 spirally circumscribing the balloon. The scoring elements 32 may continuously circumscribe the balloon or comprise a plurality of segments. The scoring elements 32 may have a variety of configurations, often being a thin structure in the form of a wire or slotted tube having a circular, square, or other cross-sectional geometry. Preferably, the scoring elements 32 will comprise a scoring edge, either in the form of a honed blade, a square shoulder, or the like.

The scoring structure 28 is preferably composed of an elastic material, more preferably a super elastic material, such as nitinol, stainless steel or a combination thereof. Scoring structure 28 may also be made of other metals such, cobalt-chromium alloy, titanium, and the like. Alternatively, scoring structure 28 may be a polymeric spiral, or made of another elastic material. When the balloon 12 is inflated, the scoring structure 28 elastically expands over the expanded balloon. Upon deflation, the scoring structure 28 will elastically close to its original non-expanded configuration, thus helping to close and contain balloon 12.

The compliance of the balloon 12 and scoring structure 28 should be chosen to assure uniform expansion of the balloon substantially free from “dog-boning” as the combined structure expands within a lesion. If a compliant or a semi-compliant balloon is used and the compliance of the scoring element was not matched to comply with the properties of the balloon, the expansion of the balloon-scoring element system will not be uniform. This non-uniformity may impair the efficacy of the scoring catheter and, in some cases, may result in poor performance. For example, under given pressure, certain parts of the balloon will be able to expand while other parts will be constrained by excessive resistance of the scoring elements.

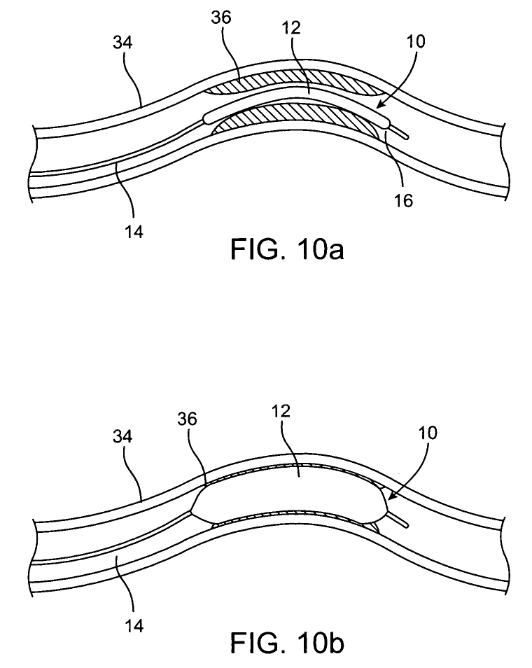

Reference is now made to FIGS. 10a and 10b, which are schematic illustrations of balloon catheter 10 in accordance with embodiments of the invention. Prior to insertion into the vessel, balloon 12 is folded along the helical fold lines into a compressed configuration. As illustrated in FIG. 10A, balloon catheter 10 is then inserted in its compressed configuration into the vascular system, for example, using a conventional catheter procedure, to a region of stenotic material 36 of blood vessel 34. (The term “stenotic” is used herein to refer to the vascular lesion, e.g., the narrowed portion of the vessel that the balloon is meant to open.) At the stenotic area, the radially expansible dilatation balloon 12 is inflated, for example, by liquid flow into the balloon from catheter body 14, as illustrated in FIG. 10B. After treatment of the stenotic area, the balloon is deflated so that the folds collapse onto the fold lines into the helically compressed state, wherein the catheter is either advanced to another treatment site or removed from the lumen. Because the fold lines are permanent, the fold lines remain on the balloon after the balloon has been delivered, inflated, and deflated.

If a scoring structure (not shown) is used, helical elements 32 widen on the inflated balloon 12. On inflation, the dilatation balloon 12 together with the helical scoring structure 28 is pressed against the walls of blood vessel 34. The pressing of scoring structure 28 against the walls of blood vessel 34 causes scoring in the stenotic area. Scoring structure 28 narrows when deflating the balloon 12, thus the balloon catheter 10 is narrowed and may be readily retrieved from blood vessel 34. The deflation profile of the balloon 10 is low and mainly circular.

Referring now to FIG. 11, a spiral folding device 50 is disclosed for fabricating spiral folds on a radially expansible balloon for a balloon catheter. The spiral folding device 50 generally comprises a base 52 connected to a mounting fixture 54 adapted to receive the balloon. Spiral folding device 50 further has an advancement means 70 for advancing the balloon relative to the mounting fixture 54. At least one scoring element 58 is mounted to the mounting fixture. In typical operation, the number of scoring elements corresponds to the number of helical folds to be fabricated, and the scoring elements converge on opening 66 through which the balloon is advanced so that the scoring elements create a plurality of scores extending helically along the outer surface of the balloon.

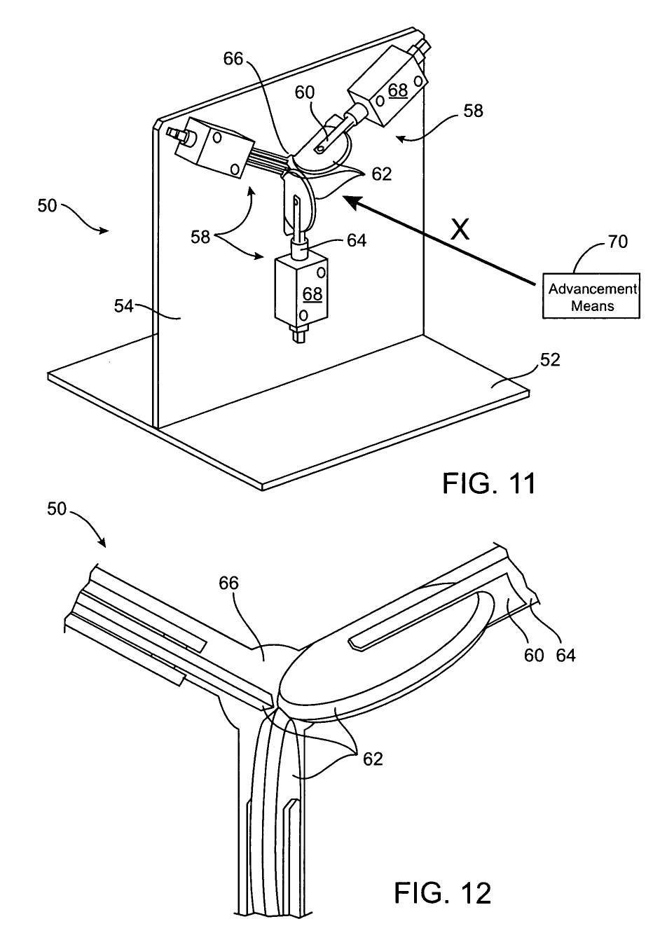

As illustrated in FIGS. 11 and 12, three scoring elements 58 are mounted to the mounting fixture 54. However, any number of scoring elements can be configured. Generally, the mounting fixture 54 has between two and five scoring elements 58, and preferably between three and four scoring elements. The scoring elements 58 may have a variety of particular configurations, and may comprise any means for permanently creating a fold line in the outer surface of the balloon, for example: scoring blades, lasers, ultrasonic emitters, radiofrequency (RF) emitters, or resistive heaters.

Where scoring blades 62 are used, the fixture further comprises an opening 66, with the scoring blades 62 each positioned to converge on the opening 66 so that the balloon may be advanced through the opening 66 to create fold or score lines on the balloon. In a preferred embodiment, the scoring blades comprise individual scoring discs 62, each rotatably mounted on a fork 60 so that the discs roll along the outside surface of the balloon as it is advanced past the opening 66. This configuration allows the balloon to be scored without scraping the surface of the balloon. Each fork 60 is fastened to the mounting fixture via an adjustable collar 64. The adjustable collar 64 allows the forks to orient the scoring blades in a variety of configurations. For example, the scoring blades 62 may be aligned to be concentric so that the scores emanate from the same point, or the blades may be aligned eccentrically to create score lines emanating at different points.

In general, the advancement means 70 comprises a linear actuator to axially advance the balloon 12 along the axis X, through the opening 66 and past the scoring elements 62. However, the advancement means may comprise any means for axially displacing the balloon catheter along its axis, such as manual insertion by hand. In some embodiments, the actuator also comprises a rotational means to rotate the balloon about its axis as it is being advanced through the fixture. The rate of rotation and axial displacement may be both controlled according to a specified helical or spiral pattern and spacing between scores.

In another embodiment, the scoring blades 62 are canted so as to force the balloon to rotate as it is advanced through the opening 66 of the fixture 54. Collar 64 can be loosened to allow the fork 64 to cant scoring discs 62 at an angular orientation out of axis with the advancement of the balloon. This rotation causes the scoring elements to create helical fold lines as the balloon is advanced through the opening without requiring the actuator to rotate the balloon as it is advanced. The catheter body 14 may be rotatably mounted on the actuator so that it freely rotates about the axis of the catheter body as the balloon is advanced.

In an alternative embodiment (not shown), the mounting fixture 54 is rotatably mounted to the base 52, and the base further comprises a means for rotating the fixture at a predetermined rate. In this configuration, the balloon is pulled axially through the fixture at a predetermined speed, thereby creating helical fold lines in the balloon as it is advanced through the rotating fixture.

Preferably, the scoring elements 58 comprise heating means 68 to heat the scoring blades to facilitate permanently engraving the fold lines into the balloon. The heating means 68 may comprise any heat source such as a resistive heater, RF energy, ultrasonic energy, or the like. The heating source may also provide means for controlling and maintaining the temperature of the scoring blades.

In one method of the present invention, the balloon 12 may be inflated to a low positive pressure prior to advancing the balloon past the scoring elements, and deflated as the balloon passes through the fixture. Generally, the balloon 12 may have an internal pressure ranging between 15 psi and 400 psi prior to insertion, and preferably between 15 psi and 50 psi. When the balloon is inflated to a positive pressure prior to insertion, the pressure is generally decreased at a rate of between 1 psi/sec and 150 psi/sec as it is advanced past the fixture.

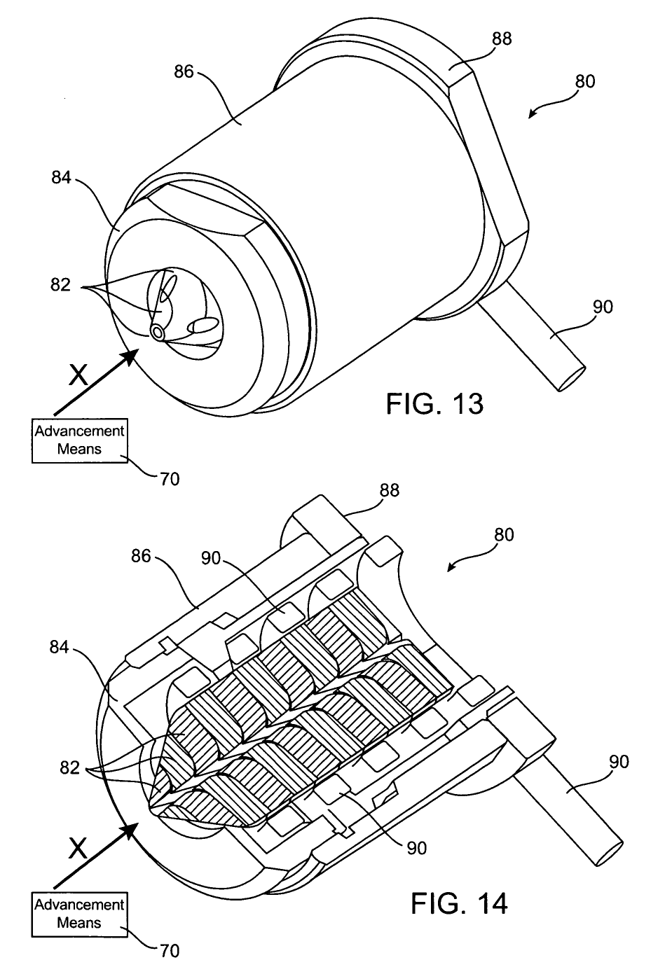

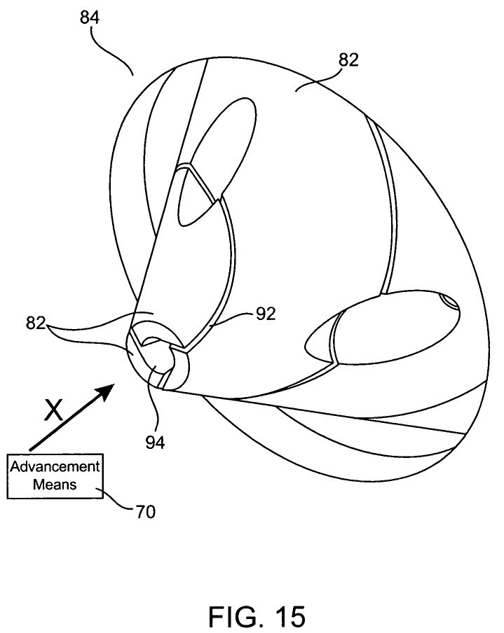

FIGS. 13-15 illustrate an alternative device comprising a helical folding press 80 for fabricating a radially expansible balloon for a spirally folded balloon catheter. The press 80 has a plurality of helical plates 82 each having mating helical surface, wherein the mating helical surfaces are placed adjacent to each other to form a at least one helical gap 92. The number of helical plates 82 and corresponding helical gaps 92 determine the number of helical folds to be placed on the balloon. One or more folds may be created with press 80, and generally two to five helical folds. The helical plates 82 axially converge on aperture 94 which is sized to accommodate the catheter body 14 to which the balloon 12 is mounted.

Helical folding press 80 further comprises a support 90 used for the holding the helical plates together. The support 90 is preferably adjustable to permit the size of the helical gaps 92 to be modified to facilitate insertion of the balloon catheter into the press. The support 90 may also provide means for applying pressure to press the helical plates 82 together. In use, a portion of the balloon 12 may be inserted along axis X between the helical plates 82 and pressed to form at least one fold line extending helically along an outer surface of the balloon. The support may comprise any means for applying pressure including a spring, threaded collar, clamp, vise etc. Generally, the support 90 and helical plates 82 rest inside casing 86 so that a portion of the helical plates protrude outside of aperture fitting 84, which is threaded into one side of the casing 86.

Ideally, the helical plates are heated with a heating means (not shown) while pressing the spiral folds in the balloon. The heating means may comprise any heat source such as a resistive heater, RF energy, ultrasonic energy, or the like. The heating source may also provide means for controlling and maintaining the temperature of the scoring blade.

Similar to the spiral folding device 50, the balloon 12 may be inflated to a low positive pressure prior to advancing the balloon into the press 80, and deflated as the balloon passes through the press. Generally, the balloon 12 may have an internal pressure ranging between 15 psi and 400 psi prior to insertion, and preferably between 15 psi and 50 psi. When the balloon is inflated to a positive pressure prior to insertion, the pressure is generally decreased at a rate of between 1 psi/sec and 150 psi/sec when being pressed.

Helical folding press 80 may also be used in conjunction with spiral folding device 50 to further set the spiral folds into the balloon. In such a configuration, the balloon catheter would first be scored with folding device 50, and then inserted into helical folding press 80 to heat and press the spiral folds. The balloon catheter may then be placed in an over tube (not shown) as the balloon is cooling to compress the folds against the catheter body so that the folds remain in the compressed state.

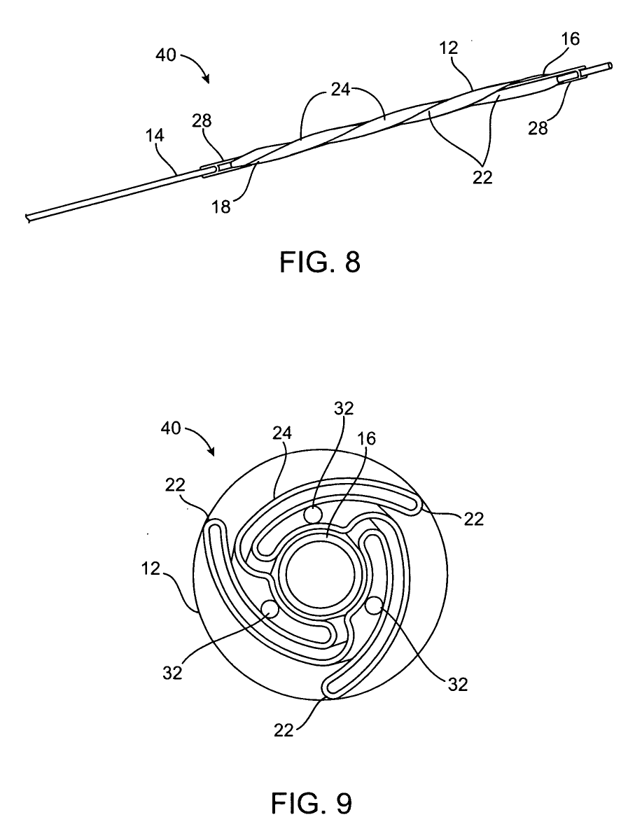

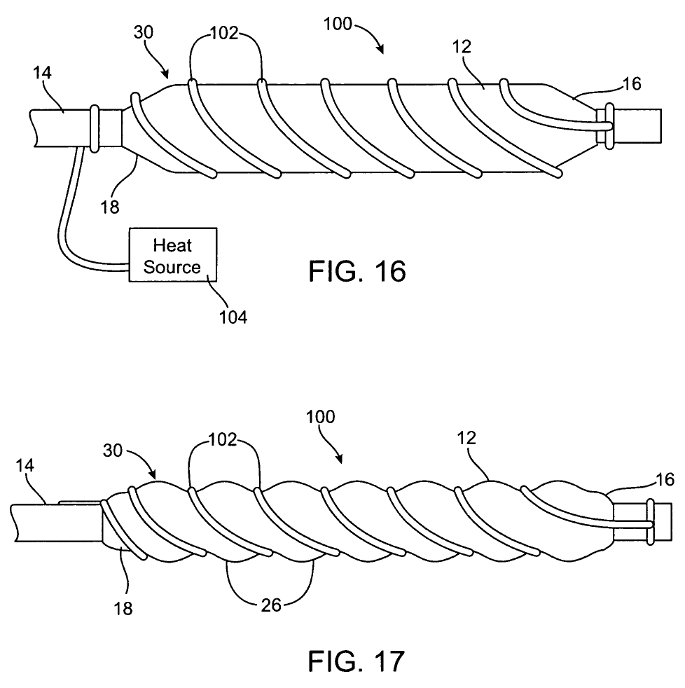

In another embodiment shown in FIGS. 16 and 17, a spiral folding cage 100 may be used to set the spiral folds into the balloon. The spiral folding cage 100 comprises an expansible cage having at least one helical segment 102. The number of helical segments corresponds to the number of helical fold lines to be fabricated. Generally cage 100 has two to five helical segments to create the helical fold lines, and preferably three to four segments. The cage may comprise wire, spiral die, skeleton or other metallic structure. Ideally, the cage is made of an expandable, super-elastic material such as nitinol, stainless steel, or other memory material. In some embodiments, the cage 100 may be a scoring structure that is used to score a region of stenotic material of a blood vessel, as described with reference to the illustrations in FIGS. 3, and 7-9.

The spiral folding cage 100 is further coupled with heat source 104 for heating the helical segments 102. The heat source may comprise any heating means such as a resistive heater, RF energy, ultrasonic energy, or the like. The heating source may also provide means for controlling and maintaining the temperature of the scoring blade.

In one embodiment, the balloon is inflated inside the cage so that the balloon contacts at least one helical segment of the cage 100. Heat is then applied to the cage 100 by heating source 104, and the cage 100 is radially compressed while deflating the balloon to create one or more fold lines along the helical segments 102. Generally, the balloon is inflated to an internal pressure ranging between 15 psi and 400 psi, and preferably between 15 psi and 50 psi. The pressure is typically decreased at a rate of between 1 psi/sec and 150 psi/sec as the cage is being compressed.

After the helical fold line(s) are set, the compressed cage and balloon may be inserted into an over tube (not shown) having an inner diameter such that the balloon is folded over onto the helical fold line(s). Alternatively, the balloon may be rolled so that the balloon folds helically along the fold line.

It will be appreciated by persons skilled in the art that the present invention is not limited to what has been particularly shown and described hereinabove. Alternate embodiments are contemplated that fall within the scope of the invention.

1. A balloon catheter having a spirally folded balloon comprising:
a catheter body having a proximal end and a distal end; and
a radially expansible balloon near the distal end of the catheter body, the balloon comprising a proximal end, a distal end, and at least one permanent fold line formed on the balloon prior to folding, the fold line extending helically along at least a portion of the surface of the balloon.

2. A balloon catheter as in claim 1, wherein the radially expansible balloon comprises two to five helical fold lines extending helically along at least a portion of the surface of the balloon.

3. A balloon catheter as in any one of claims 1 or 2, wherein each fold line is parallel to each other.

4. A balloon catheter as in claim 3, wherein the fold lines are equally spaced apart.

5. A balloon catheter as in any one of claims 1 or 2, wherein the at least one fold line comprises a groove in the balloon.

6. A balloon catheter as in any one of claims 1 or 2, wherein the at least one fold line comprises a crease in the balloon.

7. A balloon catheter as in any one of claims 1 or 2, wherein the balloon is folded along the preformed fold lines to form lobes.

8. A balloon catheter as in any one of claims 1 or 2, wherein the balloon is folded along the preformed fold lines to form flaps.

9. A balloon catheter as in claim 1 or 2, further comprising a scoring structure adjacent to the fold lines.

10. A balloon catheter as in claim 9, wherein the scoring structure comprises at least one scoring element spirally circumscribing the balloon.

11. A balloon catheter as in claim 10, wherein the scoring element continuously circumscribes the balloon.

12. A balloon catheter as in claim 10, wherein the scoring element comprises a plurality of segments.

13. A balloon catheter as in claim 10, wherein the scoring element comprises a wire.

14. A balloon catheter as in claim 10, wherein the scoring element is secured to an outer surface of the balloon.

15. A balloon catheter comprising:
a catheter body having a proximal end and a distal end; and
a radially expansible balloon near the distal end of the catheter body, the balloon comprising a proximal end, a distal end, and at least one recess extending helically along at least a portion of the surface of the balloon; and
at least one helical scoring structure located within the helical recess of the balloon, wherein the helical recess shields the scoring structure from exposure when the balloon is not expanded.

16. A balloon catheter as in claim 15, wherein the scoring structure comprises at least one scoring element spirally circumscribing the balloon.

17. A balloon catheter as in claim 16, wherein the scoring element continuously circumscribes the balloon.

18. A balloon catheter as in claim 16, wherein the scoring element comprises a plurality of segments.

19. A balloon catheter as in claim 16, wherein the scoring element comprises a wire.

20. A balloon catheter as in claim 16, wherein the scoring element is secured to an outer surface of the balloon.

21. A method of folding a balloon on a balloon catheter for insertion in a body lumen, the method comprising:
providing a balloon having at least one fold line formed in a wall of the balloon and extending helically along the outer surface of the balloon; and
folding the balloon along the at least one helical fold line.

22. A method as in claim 21, wherein the at least one fold line is created by scoring the balloon.

23. A method as in claim 22, wherein scoring the balloon comprises:
advancing the balloon relative to a fixture having at least one scoring element; and
scoring the balloon to create a score that extends helically along an outer surface of the balloon.

24. A method as in claim 23, wherein the balloon is advanced relative to a stationary fixture.

25. A method as in claim 23, wherein the fixture is advanced relative to a stationary balloon.

26. A method as in claim 23, wherein the fixture has between two and five scoring elements.

27. A method as in claim 26, wherein the scoring elements comprise lasers.

28. A method as in claim 26, wherein the scoring elements comprise scoring blades.

29. A method as in claim 28, wherein the fixture further comprises an opening, the scoring blades positioned to converge on the opening, wherein the balloon is axially advanced through the opening to score the balloon.

30. A method as in claim 29, wherein the scoring blades individually rotate about a mounting axis of each scoring blade as the balloon is advanced through the opening.

31. A method as in claim 30, wherein the balloon is rotated as it is advanced through the opening.

32. A method as in claim 28, further comprising heating the scoring blades.

33. A method as in claim 30, wherein the scoring blades are canted so as to force the balloon to rotate as it is advanced through the opening of the fixture.

34. A method as in claim 31, wherein folding the balloon along the helical fold line comprises inserting the balloon into a press.

35. A method as in claim 34, further comprising heating the press.

36. A method as in claim 23, wherein the balloon is inflated to a predetermined pressure prior to advancing the balloon relative to a fixture, and wherein the balloon is deflated at a predetermined rate as it is being scored.

37. A method as in claim 36, wherein the balloon is inflated to a range between 15 psi and 400 psi.

38. A method as in claim 37, wherein the balloon is deflated at a rate in the range between 1 psi/sec and 150 psi/sec.

39. A method as in claim 21, wherein the at least one fold line is created by permanently creasing the balloon.

40. A method as in claim 39, wherein permanently creasing the balloon comprises:
advancing a portion of the balloon into a press comprising a plurality of helical folding plates, the helical plates positioned adjacent to each other to form one or more helical gaps, wherein the portion of the balloon is positioned in the one or more helical gaps between the helical plates; and
pressing the plates together to form at least one fold line extending helically along an outside surface of the balloon.

41. A method as in claim 40, wherein the press comprises two to five helical folding plates to create a plurality of fold lines extending helically along an outside surface of the balloon.

42. A method as in claim 41 further comprising an expandable support, wherein the folding plates are held adjacent to each other by the expandable support.

43. A method as in claim 42, wherein prior to advancing the balloon into the press, the expandable support is sufficiently expanded to allow the balloon to be positioned between the mating surfaces of the helical folding plates.

44. A method as in claim 42, wherein pressing the plates together comprises compressing the expandable support.

45. A method as in claim 42, wherein the balloon is inflated to a predetermined pressure prior to advancing the balloon relative to a fixture, and wherein the balloon is deflated at a predetermined rate as it is being scored.

46. A method as in claim 45, wherein the balloon is inflated to a range between 15 psi and 400 psi.

47. A method as in claim 45, wherein the balloon is deflated at a rate in the range between 1 psi/sec and 150 psi/sec.

48. A method as in claim 21, wherein the at least one helical fold line is created by heating a portion of the balloon in a helical pattern.

49. A method as in claim 48, wherein heating the balloon comprises:
providing an expansible cage having at least one helical segment, wherein the number of helical segments corresponds to the number of helical fold lines to be fabricated;
inflating the balloon inside the cage so that the balloon contacts the at least one helical segment;
heating the cage; and
radially compressing the cage while deflating the balloon to create at least one helical fold line along the at least one helical segment.

50. A method as in claim 49, wherein the cage has two to five helical segments to create the helical fold lines.

51. A method as in claim 50, wherein the helical segments comprise wire.

52. A method as in claim 50, further comprising inserting the compressed cage and balloon into a tube having an inner diameter such that the balloon is folded over onto the helical fold line when placed in the tube.

53. A method as in claim 50, further comprising rolling the balloon so that the balloon folds helically along the fold line.

54. A method of treating a body lumen of a patient, the method comprising:
providing a catheter having a balloon with at least one fold line formed in the wall of the balloon and extending helically along the outer surface of the balloon,
inserting the catheter in its helically-folded state into the body lumen;
advancing the catheter to a treatment site within the lumen; and
inflating the balloon to engage a wall of the lumen to treat the lumen.

55. A method as in claim 54, further comprising:
deflating the balloon so that the at least one fold collapses into the helically compressed state to disengage the wall of the lumen; and
removing the catheter from the lumen

56. A device for fabricating a radially expansible balloon for a balloon catheter, the balloon having at least one helical fold, the device comprising:
a base,
a mounting fixture connected to the base, the mounting fixture adapted to receive the balloon;
means for advancing the balloon relative to the mounting fixture; and
at least one scoring element mounted to the mounting fixture, wherein the number of scoring elements corresponds to the number of helical folds to be fabricated; and
wherein the at least one scoring element converges on the balloon as it is advanced so that the scoring element creates at least one score extending helically along the outer surface of the balloon, the helical score allowing the balloon to be helically folded along the score.

57. A device as in claim 56, wherein the fixture has between two and five scoring elements.

58. A device as in claim 57, wherein the scoring elements comprise lasers.

59. A device as in claim 57, wherein the scoring elements comprise scoring blades.

60. A device as in claim 59, wherein the fixture further comprises an opening, the scoring blades positioned to converge on the opening, wherein the balloon is advanced through the opening to score the balloon.

61. A device as in claim 60, wherein the scoring blades comprise discs rotatably mounted on the fixture, and wherein the discs roll along the outside surface of the balloon as it is advanced past the opening.

62. A device as in claim 61, wherein fixture is rotatably mounted to the base, and wherein the base further comprises a means for rotating the fixture at a predetermined rate.

63. A method as in claim 61, wherein the scoring blades are canted so as to force the balloon to rotate as it is advanced through the opening of the fixture.

64. A device as in claim 61, further comprising means for heating the scoring blades.

65. A device for fabricating a radially expansible balloon for a balloon catheter, the balloon having at least one helical fold, the device comprising:
a plurality of helical plates placed adjacent to each other to form at least one helical gap;
a support for the holding the helical plates together, the collar being adjustable to permit the size of the at least one helical gap to be modified; and
means for applying pressure to press the helical plates together, wherein a portion of the balloon may be inserted between the helical plates and pressed to form at least one fold line extending helically along an outer surface of the balloon, and wherein the balloon may be spirally folded along the fold line.

66. A device as in claim 65, further comprising means for heating the plates.

67. A device as in claim 65, wherein adjusting the collar provides means for applying pressure to the press.

68. A device for fabricating a radially expansible balloon for a balloon catheter, the balloon having at least one helical fold, the device comprising:
an expansible cage having at least one helical segment, wherein the number of helical segments corresponds to the number of helical fold lines to be fabricated; and
a heat source coupled to the cage;
wherein the heat source heats the cage to create the at least one helical fold line.

69. A device as in claim 68, wherein the cage has two to five helical segments to create the helical fold lines.

70. A device as in claim 69, wherein the helical segments comprise wire.

71. A device as in claim 69, wherein the caged is configured to fit around the circumference of the balloon so that the helical segments contact the balloon when the balloon is in an inflated configuration, and wherein the cage collapses with the balloon when the balloon is deflated.

72. A device as in claim 71, wherein the cage further comprises a scoring structure that is delivered with the balloon catheter for treatment of a body lumen.

73. A device as in claim 68, further comprising an over tube, the over tube having an inner diameter such that the balloon is folded over onto the at least one helical fold line when placed in the tube.
